(19) United States
(12) Patent Application Publication
(10) Pub. No.: US 2008/0220066 A1
(43) Pub. Date: Sep. 11, 2008

(54) Anti-Viral Topical Gel Formulations Containing a Diuretic Such as Furosemide and/or a Cardiac Glycoside Such as Digoxin

(75) Inventors: Christopher Hartley, West Midlands (GB); Ian Stuart Pardoe, West Midlands (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HENDERSON MORLEY PLC, Birmingham, West Midlands (GB)

(21) Appl. No.: 12/065,581

(22) PCT Filed: Aug. 22, 2006
(86) PCT No.: PCT/GB06/03146

§ 371 Date: Apr. 16, 2008

(30) Foreign Application Priority Data
Sep. 2, 2005 (GB) 0517840.5

(51) Int. Cl. A61K 9/00; A61K 31/195; A61K 31/192; A61K 31/7048; A61K 31/341; A61P 31/12; A61P 31/20

(52) U.S. Cl. 424/486; 424/400; 514/562; 514/571; 514/26; 514/471; 424/484

(57) ABSTRACT

An anti-viral topical gel formulation comprising at least one loop diuretic and/or cardiac glycoside in a gel carrier medium, said formulation capable of transdermal delivery of the said diuretic and/or glycoside.

This invention relates to topical formulations, in particular those that are useful in the treatment of viral infections.

Viruses are intracellular parasites wholly dependent upon the infected host cell for survival. The earliest known anti-viral drugs were cytotoxic drugs, such as those used in cancer chemotherapy. It was postulated that these inhibitors of host cell metabolism would have an adverse effect upon the lifecycle of the virus. In the non-cancer patient cytoxic drugs were too toxic to the host to be of benefit, and as a consequence their use as anti-viral treatments was severely restricted.

Drugs which target metabolic pathways peculiar to a virus have since been sought but with only limited success. One successful drug, acycloguanosine and its derivatives has been developed. Its clinical usefulness however is limited to a narrow spectrum of virus infections e.g. α-herpes viruses. There are many virus infections in man for which no effective chemotherapies exist.

Anti-viral chemotherapy is further hindered by the development of drug resistance. Following the widespread use of aciclovir for the treatment of herpes simplex virus infections; resistant strains carrying mutations in the thymidine kinase gene and the polymerase gene, are readily found.

We have now developed formulations useful in treatment of a broad spectrum of viral infections. Low incidence of drug resistance is one prospective benefit. Conventional antiviral drugs, nucleoside analogues and their derivatives and protease inhibitors for example, are intimately associated with biosyntheses necessary for viral replication. The present formulations however, intervene discretely, modifying intracellular ion concentrations and their distribution and depriving viral DNA synthesis of essential co-factors. In application and use of the present formulations there is therefore intentional, but minimal and reversible suppression of host cell metabolism to a point where normal cellular function proceeds, but viral reproduction does not.

HPV (human papillomavirus) infection is one of the most prevalent sexually transmitted pathogenic diseases in the world. It is estimated that approximately 5.5 million new cases of genital HPV infections occur each year in the US alone.

Human papilloma viruses are a group of DNA tumour viruses which can induce neoplastic proliferation of human epithelial cells. More than 120 HPV types have been described, about one-third of them spread through sexual contact. Low-risk types of HPV can cause conditions such as genital warts, skin warts and veruccae (plantar warts). Other high-risk types of HPV can cause cervical and anal dysplasias, potentially leading to the life-threatening disease of cervical and anogenital carcinoma.

The HPV is accountable for the development of a spectrum of hyperproliferative diseases affecting the epithelia.

Genital warts, for example, are one form of such hyperproliferative disease, spread by sexual contact and are very contagious. Although current treatments sometimes can eliminate the warts, the elimination is rarely permanent as the warts often reappear after treatment within 3 to 6 months. In cases where larger warts are present, surgery under general anesthesia may be required.

Current clinical treatments for HPV infections involve lesion destruction. These procedures include excision using scalpel, laser, freezing, or lesion ablation with toxic agents applied topically and/or intralesionally (trichloroacetic acid, phenol, salicylic acid, podophyllin, 5-fluorouracil, acyclic nucleotides and podofilox), as well as photo-dynamic therapy.

Additional strategies including locally applied immune modulators such as imiquimod and interferon have been tried, however recurrence rates are very high (usually greater than 50%), and subclinical infections often go undetected and untreated. These latter infections may be reactivated by a variety of poorly characterised events and agents such as environmental carcinogens and/or co-factors, UV-irradiation, hormones, wounding, immune suppression and other STD agents to produce new active clinical disease.

It is clear that the current treatments which can be painful and leave scars are unsatisfactory and there is an urgent need to develop drugs with greater efficacy and specificity, especially since duration of current treatments may last many weeks to months.

The widest anti-viral treatment for herpes simplex labialis is aciclovir. However, studies with topical aciclovir have produced varied results according to the formulation used. Treatment costs for aciclovir and other nucleoside analogues are relatively high.

HPV strains are transmitted via direct contact through macroscopic lacerations in the epithelium allowing entry to the basal cells which are the site of infection. Many of the HPV strains lead to clinical presentation as warts.

There is no widely used topical, effective, prescription only anti-viral treatment available for genital warts. Genital warts are one of the most prevalent sexually transmitted infections (STI's). These excrescences are clinically distinct from other classes of warts, and there is no established effective treatment regime. HPV can infect skin distant from the wart.

Drug resistance is now becoming a major problem especially in individuals suffering immune compromise. For example, as aciclovir acts on a single metabolic event, viruses are able to overcome the effects of this drug with a single gene mutation, Individuals who are suffering with immunocompromise (caused by disease or drug induced) are particularly prone to the development of severe and refractory disease. The development of resistant strains is also much more common in those who take high dose and continuous anti-viral treatment. Current treatments do not entirely eradicate the HSV infection.

The current options available for treatment of common, plantar and genital warts can be broadly divided into two groups: 1. Antiwart therapies, which aim to mechanically, remove the infected lesion, and 2. Antiviral interventions (direct or indirect that attempt to eradicate the underlying cause). Antiwart therapies are usually first line treatment for most non-genital warts and some genital warts. There are numerous antiwart options available to the patient, including non-chemical intervention such as surgery and cryotherapy, as well as compounds such as; podophyllin, podofilox, bleomycin, salicylic acid, formaldehyde, glutaraldehyde, bichloroacetic acid, and trichloroacetic. The goal of any intervention is to remove the warts, whilst doing as little damage as possible to the surrounding healthy skin. Each of the above agents seek to destroy the cell and the virus rather than allowing the cell to survive but not the virus, which may lead to adverse effects.

Surgical removal of warts offers rapid removal of the lesion and offers a high response rate. However, this method is associated with several disadvantages, including the potential for bleeding, scarring, and bacterial infection and the requirement of a local anaesthetic. Scarring is mainly problematic in plantar warts where even the smallest scar can cause the patient discomfort. Cryotherapy—freezing the wart with carbon dioxide snow or liquid nitrogen for 30-60 seconds is painful, and can create blisters.

Still, complete response rates of cryotherapy are not as high as with surgery and multiple sessions over a period of a number of weeks is often required.

There are also several cyto-destructive products available for non-genital warts, such as salicylic acid, glutaraldehyde and formaldehyde, which soften and destroy the infected tissue. Salicylic acid is safe, moderately effective and often the treatment of choice, but has several disadvantages including being an irritant and having a long treatment duration (approximately 3 months). This has a substantial impact on patient compliance.

Podophyllotoxin (podofilox), is an active ingredient of the less commonly used natural product podophyllin solution. It is not often used because it is a mutagen and is associated with high recurrence rates. Podophyllotoxin is one of two United States Food and Drug Administration (FDA) approved genital warts preparations for self-administration. One major problem with this treatment is limiting its application to the area of the wart and its teratogenic potential.

In summary, present antiwart treatments have an array of efficacies. No single treatment is completely effective and relapse rates, along with side effects, are high.

Antiviral therapies aim to treat the underlying infection, which would appear a more rational approach compared to antiwart treatments. Cidofovir, interferon and imiquimod indirectly, all meet these criteria. Limitations exist in the delivery of these compounds to the site of action through the keratinised skin layer of the wart.

Cidofovir, a cytidine analogue is active against a whole host of DNA viruses, and has been shown also to be effective in treating HPV diseases. The antiviral effect of cidofovir in the HPV infection is thought to be due to the initiation of apoptosis (Snoeck et al, 2001). This treatment is not currently licensed for HPV infections and is unlikely to be so because of its systemic toxicity.

Interferon (IFN), is a protein that interferes, with viral replication in eukaryotic cells. The advantages of IFN lie in its immunomodulatory, antiviral and antiproliferative properties. IFNs are the only approved anti viral drugs for benign warts. IFNs are not routinely used in the clinic. Alferon® is an intralesion injection indicated for recurrent genital warts in patients over eighteen years of age. It is composed of interferon α containing approximately 166 amino acids ranging from 16,000 to 27,000 Da, limiting their potential as a topical dosage form, which may overcome the poor side effect profile associated with them.

Imiquimod is an immune response modifier that produces interferons and cytokines in humans. Clinical trials demonstrated efficacy and safety of this drug and concluded that significant genital wart clearance was observed with 5% Imiquimod cream compared with the placebo.

We have now devised ant-viral topical gel formulations for treating DNA viral infections by interference in and possible prevention of viral reproduction. Preferably, the formulations are for use in treating a wide range of DNA viral infections—including those relating Human Papilloma virus—where no current therapy exists or where current therapies are experiencing a build up in drug resistance.

According to this invention there is provided in one aspect an anti-viral topical gel formulation comprising at least one diuretic and/or cardiac glycoside in a gel carrier medium, said formulation capable of transdermal delivery of the said diuretic and/or glycoside.

Preferred embodiments of the invention are to be found expressed within the sub claims appended hereto.

The diuretic may be one or more selected from groups of diuretics comprising loop diuretics, thiazide diuretics and/or sulphonylureas.

Furosemide, known chemically as 5-(Aminosulfonyl)-4-chloro-2-[(2-furanylmethyl)amino]benzoic acid), is a loop diuretic known also as an inhibitor of Na⁺/K⁺/2Cl symport. It is currently indicated for oedema and oliguria due to renal failure and exerts its physiological effect by inhibition of the transport of chloride ions across cell membranes. The thick segment of the ascending limb of the loop of Henle is primarily the site of action for its diuretic effect, the mechanism involves binding to Na⁺/K⁺/2Cl⁻ symporters in the thick ascending limb, and consequently promoting potassium excretion, leaving cells depleted of intracellular potassium.

Digoxin, chemically known as 3β-[O-2,6-dideoxy-β-D-ribo-hexopyranosyl-(1,4)-O-2,6-dideoxy-β-D-ribo-hexopyranosyl-(1,4)-2,6-dideoxy-β-D-ribo-hexopyranosyl)oxy]-12b, 14-dihydroxy-5β,14β-card-20(22)-enolide, is a cardiac glycoside obtained from the leaves of Digitalis lanata. It contains a steroid nucleus, with an unsaturated lactone essential for activity at the C17 position, and one or more glycoside residues at C3.

The pharmacological activity of Digoxin is related to its ability to specifically bind to a site on the extracytoplasmic face of the a subunit of the enzyme Na⁺, K⁺ ATPase (regulates the concentration of sodium and potassium inside cells). This selectively inhibits the cellular active transport Na⁺ and K⁺ pump, which leads to an increase in the intracellular concentrations of sodium and calcium, and a decrease in the concentration of intracellular potassium. It is currently indicated in heart failure and supraventricular arrthymias. One main limitation to its clinical use when administered orally or parentarally is the risk of side effects such as nausea and vomiting associated with toxicity as it possess a narrow therapeutic window.

Dermatological preparations are defined as formulations designed to deliver compounds to the skin surface, within the skin compartment or to a related area located in physical proximity to the site of application’. A dermatological delivery system is principally advantageous over oral delivery (most common route) as high drug concentrations can be delivered to the site of pathology avoiding systemic distribution. However, disadvantages including, potential irritation to the skin, and a slow onset of action is often observed. The present invention is seeking to provide dermatological preparation from which percutaneous absorption of preferably Digoxin and/or Furosemide to the basal layer of the epidermis can be achieved. Although absorption is largely determined by the physiochemical properties of a compound, the formulation can play an important role in determining the flux of a compound.

A gel is a material that often contains small discrete particles in which case it is defined as a two component system of semisolid nature, rich in liquid. The one characteristic feature is the presence of a continuous structure often termed a ‘thickener or thickening agent’ which provides solid-like properties. In a typical polar gel, a synthetic or natural polymer usually builds a three dimensional matrix throughout a hydrophilic liquid. If the gel contains small discrete particles, the gel is a two phase system, whereas if the gel does not appear to contain discrete particles it is referred to as a one-phase system. Two-phase systems tend to be thixotropic, i.e. semisolid on standing but liquify when shaken. Typical polymers used as gelling or thickening agents for one-phase systems include natural gums, carrageenan, agar, pectin, whereas two-phase systems contain semi-synthetic materials such as methylcellulose, hydroxyethylcellulose, hydroxypropylmethylcellulose, and carboxymethylcellulose, and synthetic polymers Carbopol® (Carbomer) and Cabosil®. Gels tend to release permeants well allowing relatively good diffusion of molecules that are not too large.

In order that the invention may be illustrated, more easily understood and readily carried into effect by those skilled in the art, reference will now be made to the accompanying drawings purely by way of non-limiting examples and in which:

FIG. 1 shows the saturated solubility of Digoxin only in various solvents at 32° C.;

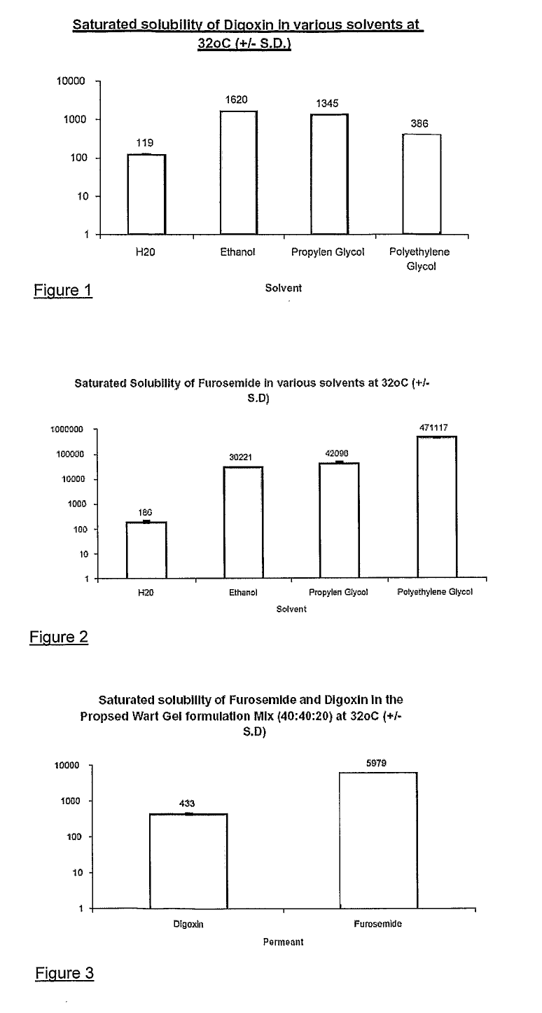

FIG. 2 shows the saturated solubility of Furosemide in the same solvents at 32° C.;

FIG. 3 indicates the saturated solubility at 32° C. of Furosemide and Digoxin in the prototype wart gel formulation comprising the co-solvent mix 40PG: 40 water: 20 urea;

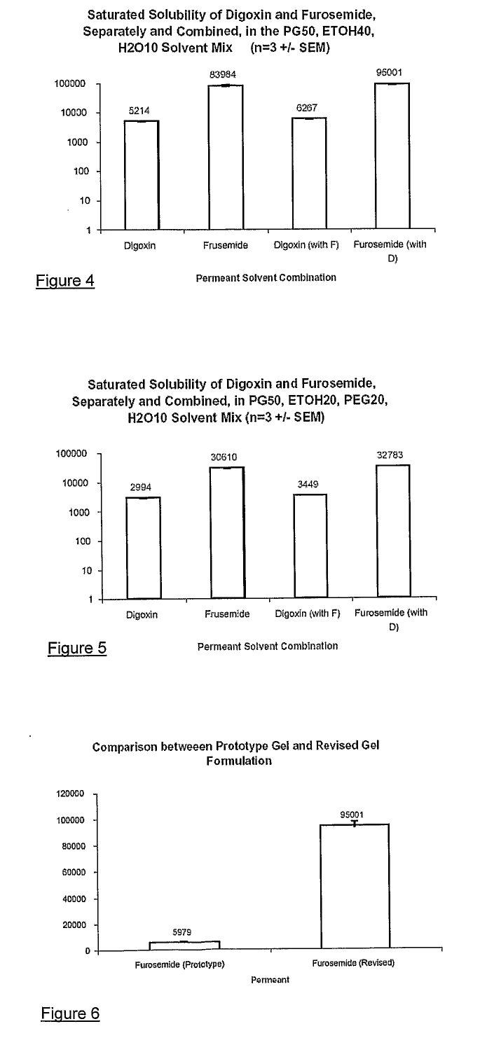

FIG. 4 indicates the saturated solubility of Furosemide and Digoxin, separately and combined, in a proposed wart gel formulation comprising a revised co-solvent mix 50PG:40 EtOH:10H₂O;

FIG. 5 shows the saturated solubility of Furosemide and Digoxin in a gel formulation comprising a co-solvent mix of 50PG: 20 EtOH: 20 PEG 400:10 H₂O;

FIG. 6 demonstrates the difference in Furosemide solubility between the prototype gel mix referred to in FIG. 3, and the revised co-solvent mix referred to in FIG. 4;

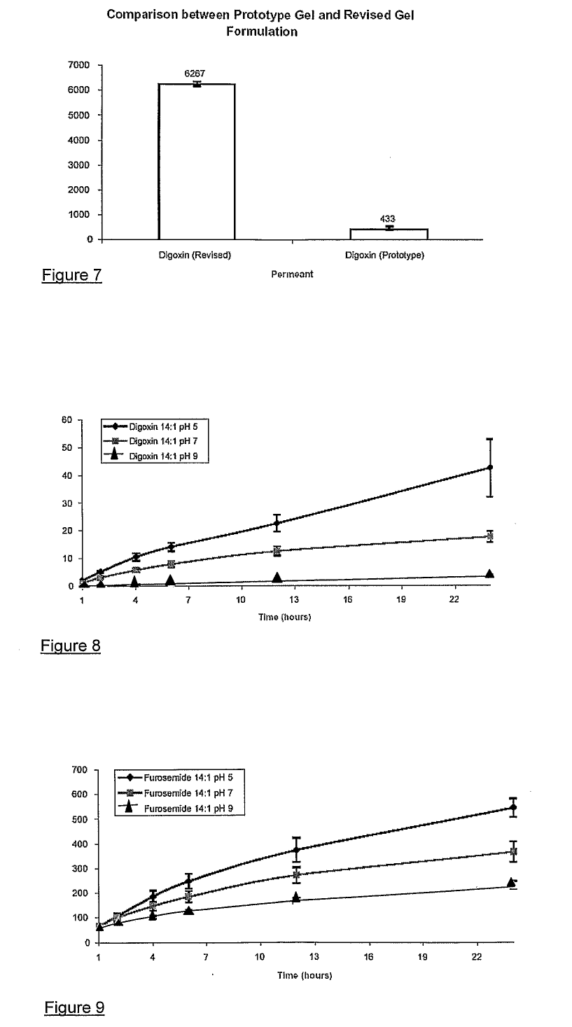

FIG. 7 demonstrates the difference in Digoxin solubility between prototype 40:40:20 co-solvent mix and the revised 50:40:10 co-solvent mix;

FIG. 8 demonstrates the effect of pH on Digoxin drug release from the gel and in particular an In-vitro release profile for Digoxin obtained from the 14:1 molar ratio gel at pH 5, 7 and 9;

FIG. 9 demonstrates In-vitro release profiles for Furosemide from the 14:1 molar ratio gel at pH 5, 7 and 9;

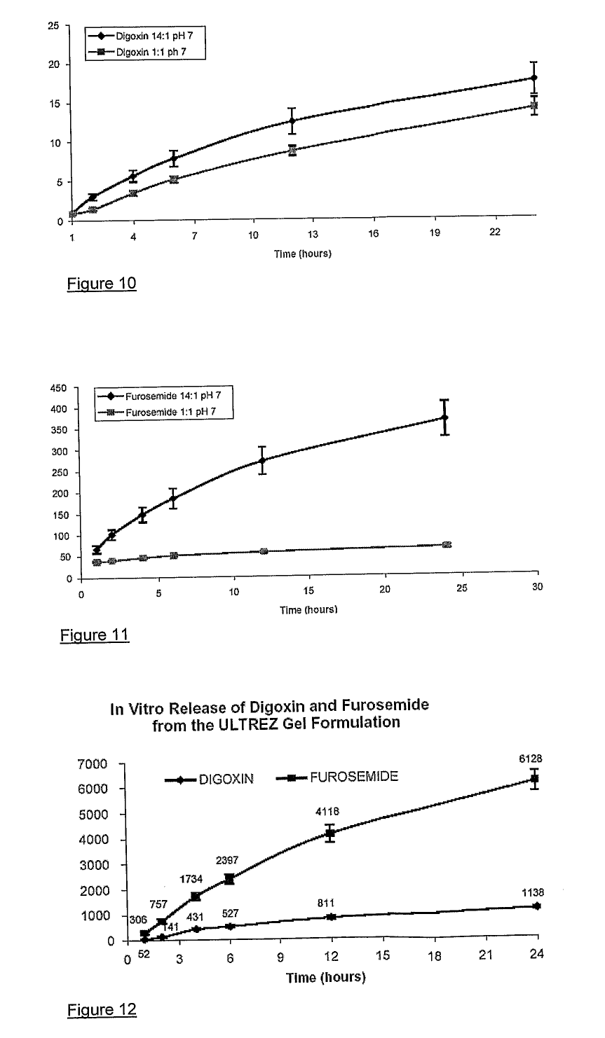

FIG. 10 demonstrates In-vitro release profiles for Digoxin from 14:1 and 1:1 carbomer gels at pH 7;

FIG. 11 demonstrates In-vitro release profiles for Furosemide from 14:1 and 1:1 (F:D) carbomer gels at pH 7;

FIG. 12 using the revised gel formulation (thickened with Ultrez Carbomer) demonstrates In-vitro release profiles for Digoxin and Furosemide;

FIG. 13 using a revised gel formulation (thickened with HPC) demonstrates In-vitro release profiles for Digoxin and Furosemide;

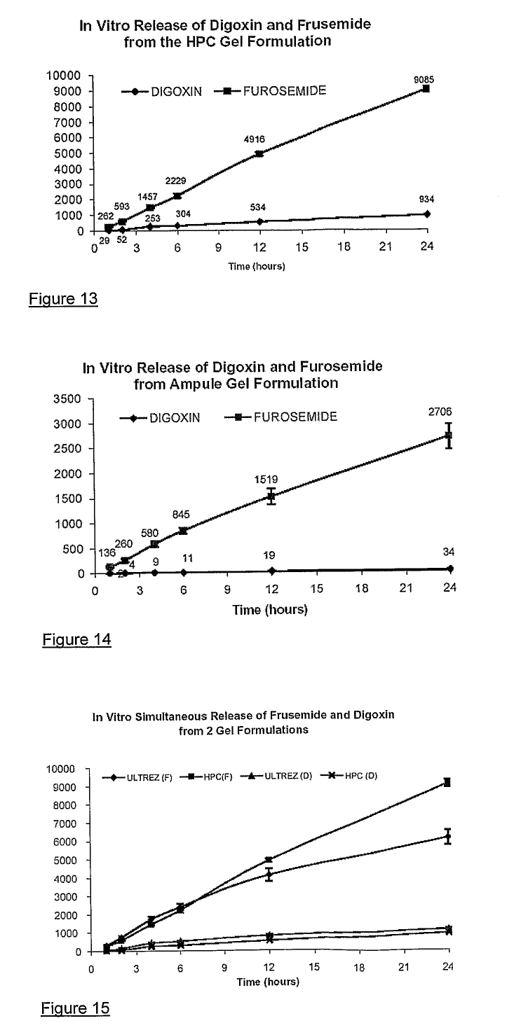

FIG. 14 using the ampule gel formulation (thickened with HPC) demonstrates In-vitro release profiles for Digoxin and Furosemide;

FIG. 15 shows a comparison of In-vitro release of Digoxin and Furosemide from gel 1:14 (D:F) at pH 5 formulations thickened with either Ultrez or HPC;

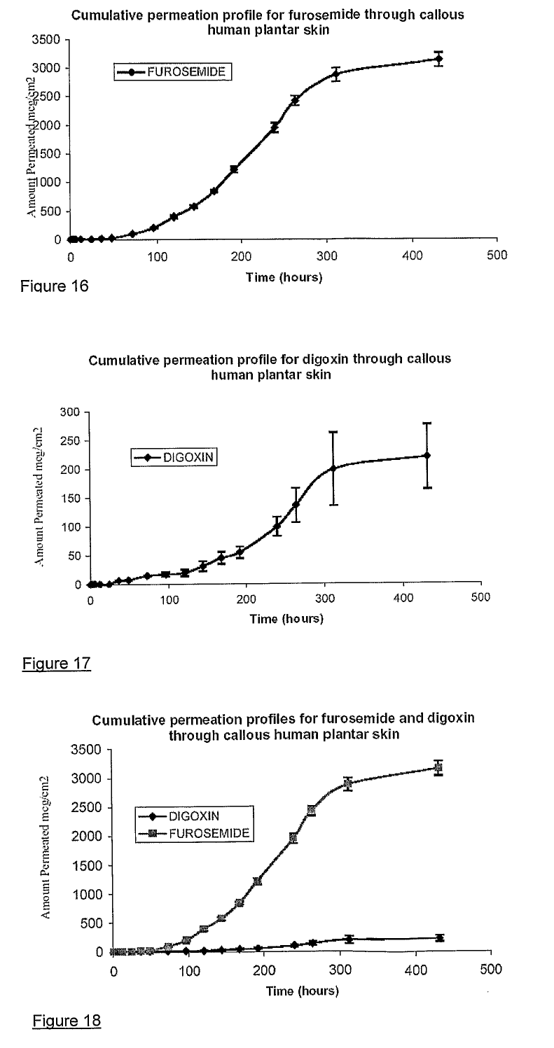

FIG. 16 shows, in relation to the prototype gel, the cumulative permeation of Furosemide through human callous plantar skin;

FIG. 17 shows, in relation to the prototype gel, the cumulative permeation of Digoxin through human callous plantar skin;

FIG. 18 shows, in relation to the prototype gel, the cumulative permeation of Digoxin and Furosemide through human callous plantar skin;

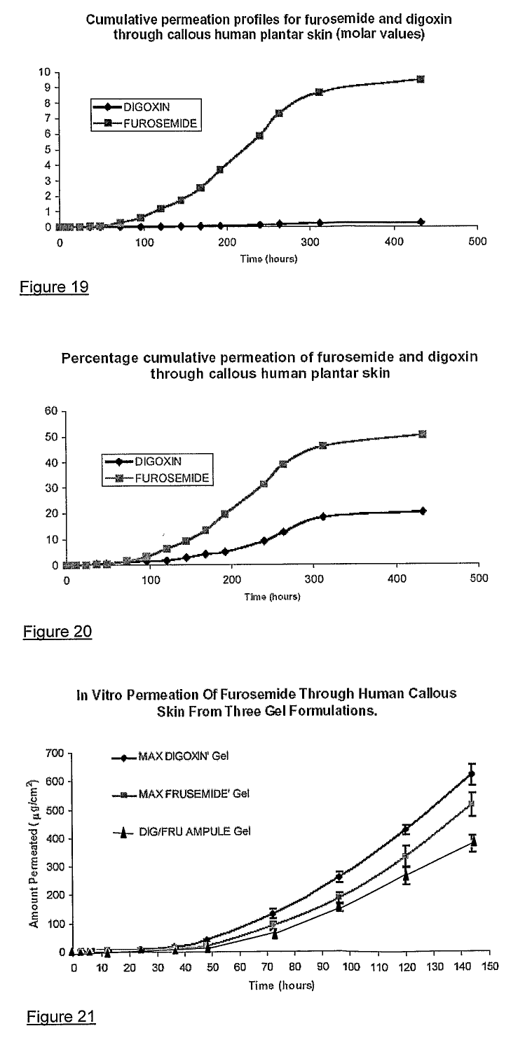

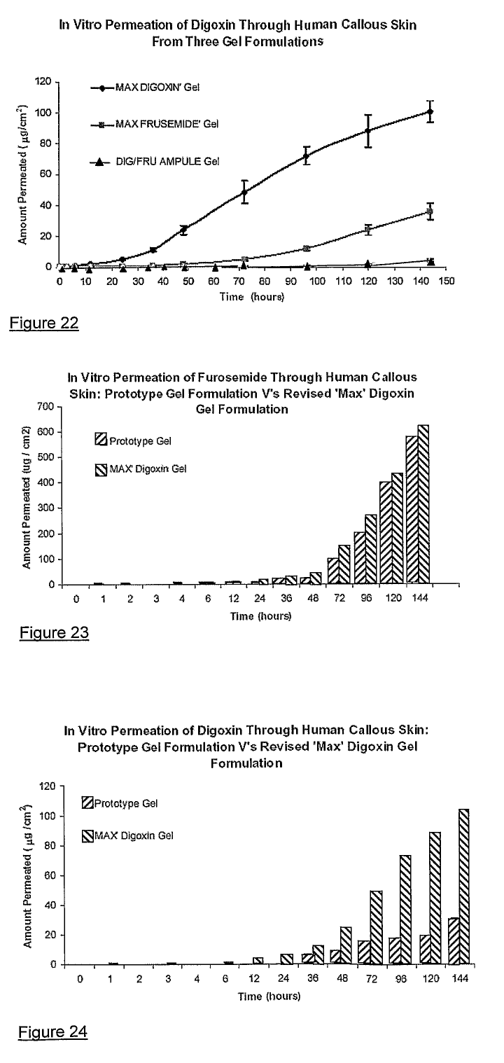

FIG. 19 shows, in relation to the prototype gel, profiles of cumulative permeation of Furosemide and Digoxin through human callous plantar skin expressed as molar amounts;

FIG. 20 shows, in relation to the prototype gel, the cumulative permeation of Furosemide and Digoxin through human callous plantar skin expressed as a percentage of the dose applied;

FIG. 21 shows In-vitro permeation of Furosemide through human callous skin from three gel formulations;

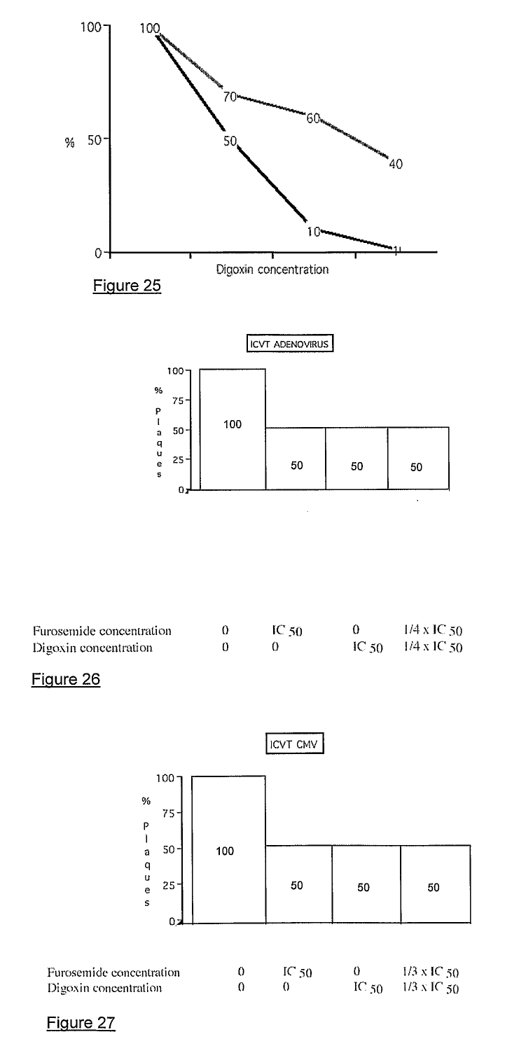

FIG. 22 shows In-vitro permeation of Digoxin through human callous skin from the same three gel formulations depicted in FIG. 21;

FIG. 23 shows In-vitro permeation of Furosemide through human callous skin: prototype gel formulation compared with revised maximum Digoxin gel formulation; and

FIG. 24 shows In-vitro permeation of Digoxin through human callous skin: prototype gel formulation compared with revised maximum Digoxin gel formulation.

It is widely recognised that the stratum corneum of human skin is the rate limiting step in terms percutaneous absorption from topical applied agents. The stratum corneum in skin areas populated with viral warts is significantly different composing of densely packed corneocytes with limited lipid intracellular pathways. This results in a stratum corneum with potentially even greater drug absorption rate limiting capabilities.

When formulating topical formulations, one approach is to chemically modify the barrier properties (making the barrier less efficient) by the incorporation of non-pharmacologically active (and ‘skin friendly’) excipients.

As was the case with each different type of anti-HPV formulations, the primary aim was to formulate topical formulations that can deliver ideally both Digoxin and Furosemide (that act synergistically) to the site of action (assumed to be the Basal Lamina as this is the site of keratinocyte replication) at efficacious amounts with minimal toxicity.

These two preferred drugs act synergistically and a primary aim is to provide topical gel formulations which deliver them together in beneficial proportions. An effective way of achieving this is to employ a ‘homogenous system’ i.e. a single phase formulation. Furthermore, structured matrices (e.g. gel) provide better active release and minimal excipient inter-reaction. The occlusive nature of a gel also assists in coating the lesion more effectively.

There are a wide range of topical formulation excipients available however the preferred ones selected took account of the following factors:
1 Degree of keratolytic activity of regular excipients.
2 Effects on ‘swelling’ of keratin.
3 Relative solubilites of the actives in the solvents bearing in mind both actives are relatively insoluble in H₂O (to keep thermodynamic activity high at relatively low concentrations thus maintaining efficacy and maintaining minimal toxicity).

### Urea (BP, EP USP)

Urea is commonly used in emollient formulations and has been shown to increase hydration of the stratum corneum at concentrations of 2-20% becoming keratolytic at 20%.

In some preferred embodiments Urea is incorporated into the gel at 20% therefore providing hydration of the stratum corneum (additional effects on swelling) whilst simultaneously exerting mild keratolytic effects.

By hydrating the pores this may also facilitate movement of the active through openings in the SC. Another advantage of incorporating this excipient is that it is a natural product of metabolism and is excreted in the urine without systemic toxicity.

### Propylene Glycol (PG) (BP, EP, USP)

PG is one of the most commonly used formulation excipients available and is frequently used at relatively low to medium concentration. The main reason for its use in topical formulations is excellent properties as a solvation agent. PG also increases water content in the stratum corneum encouraging an osmotic gradient through the stratum corneum.

If PG is used at concentrations of 40-70% it is acts as a keratolytic agent. PG is minimally absorbed and any systemic absorbance is oxidised in the liver to lactic acid pyruvic acid.

### Sterile H₂0 (BP, EP, USP)

Swelling (and associated hydration of the tissue) is deemed highly preferred to permit optimal delivery of the two actives through the otherwise densely packed corneocytes/keratin to target zone the basal layer.

### Ethanol (ETOH) (BP, EP, USP)

Ethanol is frequently used in topical formulation predominantly to improve drug solubility within the formulation. When exposed to the skin it causes dehydration, lipid mobilisation and potentially ‘skin cracking’ at relatively high concentrations. PEG Average Molecular Weight 400 (BP, EP, USP)

Polyethylene glycol 400, also known as liquid macrogol, is a water miscible vehicle, co-solvent and a humectant and may withdraw moisture from the skin. It is a clear liquid with a slight alcoholic odour having a density of 1.13 g cm⁻³ at 20° C. (water=1), and a viscosity of 43 Cs at 40° C.

PEG 400 is also known in topical formulations to improve drug solubility within the formulation. There is no scientific evidence that suggests PEG400 exhibits keratolytic activity, but there is some evidence to suggest it aids hydration of the skin.

### EXAMPLE 1

The swelling effect (i.e. absorption) of excipients ethanol (EtOH), polyethylene glycol (PEG), propylene glycol (PG) and water (H₂O) on human callous skin was determined by submersing pre-weighed skin samples in those solvents for 24 hours. The skin samples were then removed, surface solvent gently removed with tissue, and re-weighed. Results show that only water was absorbed into the skin to any great extent. For this reason water is preferably incorporated into the gel, in the form of a hydrogel. It was also found that in polyethylene glycol/water solutions, an increase in PEG resulted in a decrease in the swelling of callous skin. Incorporation of significant PEG into a gel is therefore less preferred.

### EXAMPLE 2

Propylene glycol (PG)/water solutions were made and show that PG had no undesirable effects on the swelling effects of water. There was no significant difference in swelling between a 100% water solution and a 50:50 solution of PG and water. Incorporation of 40% PG into a gel could therefore provide keratolytic action without affecting skin swelling.

### EXAMPLE 3

Literature indicates that a 20% urea solution exhibits keratolytic properties, including urea into a gel formulation is preferable for some embodiments (urea also makes gels/creams feel less greasy). By maintaining propylene glycol at 40%, excipients mixes were prepared with varying amounts of urea (up to 20%) to determine any effects on swelling. Results showed that urea had no adverse effect on the swelling (amount of uptake of water) of callous skin to any significant extent. The incorporation of 20% urea into the gel is therefore preferred in some embodiments, keeping it at a known (yet minimal) keratolytic concentration.

These results show that propylene glycol had no effects on the swelling which appears to be solely due to water. Urea has also been shown to have no undesirable effect on uptake. These results suggest that gels containing in the region of 40% water, 40% propylene glycol and 20% urea represent some preferred embodiments with dual keratolytic properties of propylene glycol and urea, together with optimum swelling properties.

When developing topical dosage forms and prior to conducting any in vitro release or permeation experiments, it is important to carry out pre-formulation studies to acquire information on the physical and chemical properties of the prospective drug and solvent candidates. The solubility of the permeant in both the vehicle and in the various phases present in the skin, among other factors, determines its relative rate of penetration.

Typically, when applied to the skin the active or permeant will be present either dissolved or dispersed in a solvent or vehicle. Although the concentration of permeant present within the skin generally controls the rate of transport, that particular concentration is dependent on the solubility (i.e. thermodynamic activity) of the permeant present in the vehicle present on the surface of the skin.

### EXAMPLE 4

Relative solubilities of Digoxin and Furosemide permeant drugs were evaluated in a series of regular solvents/excipients. The solubilities were investigated at, 32° C., the average temperature at the surface of the skin. The results were then used to determine preferred permeant solvent combination for in-vitro release and permeation to arrive at preferred gel formulations delivering optimum amounts of the two actives.

### General Procedure

Using a metal spatula, small amounts of each model permeant were separately placed into 1.5 ml vials (approximately 50 mg. occupying≈¼ volume of vial). The vials were then individually transferred onto a previously calibrated electronic balance and subsequently tared. Precisely 1 g (weight/volume w/v) of each solvent was then carefully added to the vial using a calibrated 1000 μl Gilson pipette, as the mass became closer to required 1 g required, the 1000 μl Gilson pipette was exchanged for a 200 μl and 20 μl Gilson pipette to enable accurate displacement of smaller volumes of the solvents. This procedure was carefully conducted to account for the different densities exhibited by the various solvents being studied.

Given that the solubility example was conducted at, 32° C., before solvent was added to the vial, all solvents were temperature-equilibrated at their designated temperatures in a thermostatically controlled incubator with a digital temperature reading (this was validated by placing a thermometer inside the incubator alongside the solvents). Subsequent weighing of the solvent into the respective vials containing the permeants was also carried out on an electronic balance at the appropriate study temperatures.

Negative control vials were present, containing each of the solvents with no drug added. For each permeant/solvent combination, a total of four replicates where carried out.

Once all solvents had been accurately weighed, the vials were secured into a blood cell rotator. Again this procedure was carried out at the predetermined temperature. An incubator was used to maintain a constant temperature of 32° C. The thermometer readings were periodically noted to ensure a stable consistent temperature was being maintained.

On commencement of the saturated solubility examples, the vials were periodically examined to ensure there was excess drug present within the solvent, i.e. visible solid particles or a suspension rather than a clear solution. If any vial visually showed no particles present then additional amounts of permeant where added until complete saturation was achieved for that specific permeant solvent combination was achieved. The vials were allowed to rotate for a total period of 24 hours.

Following a final visual inspection to ascertain presence of excess solid and the assumption that the equilibrium had been attained, the vials were immediately transferred to a centrifuge and spun at 12,500 rpm for 10 minutes. The centrifuge had been temperature pre-equilibrated at 32° C. By centrifuging the vials, separation of saturated permeant solvent from excess permeant was achieved, with the excess solid forming a pellet at the bottom of the vial.

750 μl (approximately 75%) of the supernatant was then transferred into another vial (pre warmed), and spun for a second time in a centrifuge at 12,500 rpm for 10 minutes. This ensured that any excess particulate matter from the pellet that could have occurred when removing the supernatant using the Gilson pipette was not present. Great care and attention was taken to ensure all equipment used including pipettes and tips were equilibrated at the corresponding temperature prior to their use to avoid any changes in permeant solubility with potential small changes in temperature. The vials were then sampled using pre-warmed pipette tips and analysed immediately by HPLC.

### EXAMPLE 5

From the results gained in the initial solubility examples of each of the permeants in each individual solvent, an additional solubility example was conducted using the same procedure as described previously, except using the following co-solvent mixes.

| | |
|---|---|
| First Co-solvent mix: 40:40:20 | Propylene Glycol:Water:Urea |
| | (Maximum Keratolytic Potential) |
| Second Co-solvent mix: 50:40:10 | PG:EtOH:Water |
| | (Maximum Digoxin Potential) |
| Third Co-solvent mix: 50:20:20:10 | PG:EtOH:PEG400:Water |
| | (Maximum Fru/Optimum Digoxin) |

FIG. 1 to 5 show the results obtained from each of the solubility tests. FIGS. 6 and 7 highlight comparisons. The solubility test results shown in FIGS. 1 to 3 indicated that both actives Furosemide (F) and Digoxin (D) were relatively insoluble in water, but their solubility increased in the presence of propylene glycol (PG). The presence of urea reduced solubility of both actives F and D.

From the solubility studies of the permeants in each individual solvent Digoxin was shown to have the highest solubility in ethanol and propylene glycol with relatively low solubility in PEG400 and water. Furosemide was shown to have significantly highest solubility in PEG400, relatively good solubility in PG and ethanol and low solubility in water.

The highest solubility of Digoxin in the presence of Furosemide in the 40:40:20 (PG:Water:Urea) co-solvent mix (prototype) was 433 and 5979 μg ml⁻¹ respectively.

### EXAMPLE 6

When applied to patients within the clinic we observed that the tissue of the patients appeared to be very hydrated, and in one case the ‘wart’ lesion disappeared completely after 16 weeks. The prototype formulation had visually appeared to generally swell and break up the lesion, although the response time suggested revisions to the formulation were necessary, by way of increasing (maximising) the level of D and F within the formulation.

It was hypothesised that although the first (40:40:20 PG:Water:Urea) co-solvent mix that had significantly greater ‘keratolytic’ potential than the second (50:40:10 PG:EtOH:Water) co-solvent mix and still appeared to enable delivery of the actives through the ‘wart’ tissue, (supported by in-vitro permeation studies) the chemical potential within the formulation may have been insufficient to diffuse to the location of the virus in efficacious amounts. It was also hypothesised that this may have been exacerbated by water in the formulation and hydrated lesions which may present an additional barrier to the ingress of the lipophilic Digoxin and Furosemide.

From the solubility studies pertaining to the second and third gel co-solvent mixes (50:40:10 PG:EtOH:Water and 50:20:20:10 PG:EtOH:PEG:Water) it was apparent that the co-solvent mix specifically designed to maximise the concentration primarily of Digoxin (50:40:10) also maximised the concentration of Furosemide (more than the gel formulation that contained PEG400). On this basis only the ‘Maximum Digoxin’ gel was subjected to in vitro release testing.

In the Maximum Digoxin second co-solvent mix the solubility of Digoxin increased from 433 μg ml⁻¹ to 6267 μg ml⁻¹ (−15 fold). The solubility of Furosemide also increased from 5979 μg ml⁻¹ to 95001 μg ml⁻¹ (˜16 fold). This unexpected increase in penetrant solubility was attributed to several factors:
incorporation of 40% ethanol.
removal of urea from the system,
reduction in the H₂O content to 8%,

Loss of urea from the formulation would be expected to reduce keratolysis within the revised gel formulation. However, to compensate for this factor, the PG content was increased to 50%.

One means of optimising the delivery of permeants into the skin from dermatological formulations is to improve the thermodynamic activity or ‘chemical potential’ of the permeant in the delivery system. Optimum release and subsequent delivery of the permeant into and across the skin can usually be obtained when the thermodynamic activity is at its highest achievable level, i.e. at saturation (=1). Being above (drug crystallisation effects) and below (lower chemical potential) the saturation level results in reduction in release from the formulation and subsequent delivery across the skin. Therefore, achieving thermodynamic activity level 1 in the topical formulation is desirable to optimising delivery of the permeants into the skin. The respective levels of Digoxin and furosemide required to achieve saturation in the various co-solvent mixes was investigated in the solubility tests. Other formulation factors must also be considered in addition to the thermodynamic for e.g. Furosemide is amphiphillic in chemical nature and so formulation pH effects could affect performance.

### EXAMPLE 7

To investigate phenomena such as this, and others, batches of several different types of gel were formulated to probe the effect these parameters may have on the in vitro release characteristics of Digoxin and Furosemide.

The effects of different types of gel thickeners for example carbomers such as Carbopol 981NF Carbopol and ULTREZ. The cellulose derivative, Hydroxypropylcellulose was also investigated as a suitable thickening agent.

In addition to the types of thickener (and the respective am required to gain a ‘desirable’ viscosity) the effects of modifying the pH of the gel to pH 5, 7 and 9 was investigated.

Finally, the effect of altering the molar ratio of the permeants to a 1:1 and 1:14 molar ratio of Digoxin:Furosemide were investigated. The rationale behind these choices is discussed herein;

### Rationale 1: Gel Formulation Containing a Saturated Solution of Digoxin and Furosemide in the 40:40:20 Co-Solvents Mix at a 1:1 Molar Ratio

The basis for this rationale was the assumption (reasonable given the known synergistic effect) that efficacy of the binary drug treatment of warts is based upon 1:1 (equimolar) delivery. This was achieved by formulating saturated solutions of Digoxin and Furosemide separately in the gel co-solvent mix (40:40:20 Water:Propylene Gycol:Urea) and then combining them at pre-determined volumes such that equimolar amounts of the two drugs were contained in the solution.

The saturation limit of Digoxin in the 40:40:20 (PG:Wata:Urea) co-solvents mix was 433 μg cm⁻³ (see saturated solubility results section). The number of moles in solution is 433/780.9=0.55 μmol cm⁻³. The saturation limit of Furosemide in co-solvents=5979 μg cm⁻³. The number of moles in solution is moles 5979/330.7=18.1 μmol cm⁻³. Therefore, for a 1:1 molar ratio we require 18.1/0.55=32.9 cm⁻³ of a saturated Digoxin solution to 1 cm⁻³ of the equivalent Furosemide solution. By combining saturated solutions of Digoxin and Furosemide at a volumetric ratio of 33:1 respectively, a solution containing both in a 1:1 molar ratio should result. (This is only true however if no drugs drop from solution on mixing—experimentally this was found to be the case). Both drugs in this case are at saturation and thus highest thermodynamic activity.

### Rationale 2: Gel Formulation Containing a Combined Saturated Solution of Digoxin and Furosemide in the 40:40:20 Co-Solvents Mix, Molar Ratio 1:14.

The basis for this rationale was to obtain maximal amounts of both drugs in the mix independently and with no control of stoichiometry. Essentially, the presence of excess of both drugs would establish equilibrium at saturation for each component in the presence of the other. Although a more ‘bucket chemistry’ approach, the end result may prove superior in performance in the clinic to the former rationale.

When excess amounts of each drug were added to the co-solvent mix subsequent HPLC analysis resulted in a respective 1:14 Digoxin:Furosemide molar ratio. This molar ratio was achieved due to the relative chemical potentials of each permeant in each others presence within the co-solvent mix. Although the molar ratio is in some ways surprisingly different to rationale 1, again each permeant will be at saturation and thus its highest level of thermodynamic activity. Furthermore, if Digoxin is the more potent of the two actives, excess Furosemide may be beneficial at the site of action sodium/potassium pump). It was hypothesised that pH variation would have minimal effect on the release of Digoxin, as it is a neutral molecule.

The following table details the different types of gels manufactured that were subsequently subjected to in vitro release testing and the proportion of each ingredient used (w/w).

| | | | | | Revised | Revised | |
|---|---|---|---|---|---|---|---|
| | | | | | ‘Max’ | ‘Max’ | F & D |
| | Prototype | Prototype | Prototype | Prototype | Digoxin | Furosemide | Ampule |
| Propylene | 40 | 40 | 40 | 40 | 50 | 50 | * |
| Glycol | | | | | | | |
| Ethanol | | | | | 40 | 40 | * |
| PEG 400 | | | | | | | * |
| Water | 40 | 40 | 40 | 40 | 10 | 10 | * |
| Urea | 20 | 20 | 20 | 20 | | | * |
| Thickener | 1.5% | 1.5% | 1.5% | 1.5% | 1.5% | 8% HPC | 8% |
| | 981NF | 981NF | 981NF | 981NF | Ultrez | | HPC |
| pH | 5 | 7 | 9 | 7 | 5 | 5 | * |
| Molar | 14:01 | 14:01 | 14:01 | 01:01 | 14:01 | 14:01 | * |
| Ratio F:D | | | | | | | |
| Co-solvent | 40:40:20 | 40:40:20 | 40:40:20 | 40:40:20 | 50:40:10 | 50:40:10 | Ampule |
| Mix | | | | | | | |
| Annotation | | | | | | | |

Empirical formulae of each of the gels.

### EXAMPLE 8

### Gel Preparation

All calculations were based on the relative proportions of the different formulation co-solvent mixes. In each different gel formulation, the thickener was added last as a percentage of the total w/w formulation mix.

The following sequence of events describes the preparation of the ‘revised maximum Digoxin’ gel. All other gels were also manufactured using the same sequence of events except using the appropriate components as described in the previous table. All gel manufacture was carried out to GLP standards. pH adjustment was carried out following addition of the thickener by the addition of appropriate amounts of NaOH or acetic acid. For those gels that contained Urea, the predetermined amounts of Urea was added following the combination of each co-solvent and before the permeants were added.

Into a clean one litre glass beaker, the ‘Max Dig’ gel formulation was prepared by firstly combining 250 g of Propylene glycol with 200 g of Ethanol and 50 g of Water (to constitute the 50:40:10 ratio). The amounts were weighed accurately on a pre-calibrated electronic balance and continually mixed with the use of a magnetic stirrer. Excess amounts of Furosemide and Digoxin were added to the co-solvent mix, to ensure a saturated solution of both Digoxin and Furosemide was obtained. The beaker was immediately sealed using para-film (to stop any solvent evaporation) and left continually stirring at room temperature over night. The resulting suspension was then centrifuged at 25,000 rpm for 20 minutes to separate excess Furosemide and Digoxin drug, from the resultant saturated co-solvent mix. The resultant saturated solution was transferred into another clean one litre beaker, which had been previously placed on an electronic balance, with care taken in ensuring no excess permeant was also transferred, and the total weight of the saturated solution recorded. Using an electronic balance, HPC (hydroxypropycellulose) was subsequently weighed that corresponded to 8.0% w/w of the total saturated solution (w/w).

Whilst the saturated co-solvent mix was agitating vigorously (with the use of an overhead stirrer), with the use of a spatula, HFC was slowly added over a period of five minutes. Following visual inspection to confirm that the HFC had been fully dispersed, the container containing the gel formulation was placed onto a blood tube rotator and left overnight to allow the HPC gel network to form. A resultant homogenous batch gel formulation was obtained. This procedure was repeated, at a smaller scale, to obtain a gel formulation consisting of 50:20:20:10 Propylene Glycol:Ethanol:PEG 400:Water co-solvent mix.

To formulate a gel from the Digoxin and Furosemide ampules, equal amounts of the Digoxin ampule (62.5 mg/ml) and the Furosemide ampule (20 mg/ml) were accurately weighed onto an electronic balance. When combining the ampules there is a reduction in the total ethanol content (as only the Digoxin ampule contains ethanol). To compensate for this (to ensure Digoxin does not precipitate out when combined with Furosemide) for each 2 g of Digoxin that was combined with 2 g of Furosemide 200 μl of ethanol was also added. Again 8% w/w of HPC was added and the solution was left to form a gel following the same procedures as described above.

### EXAMPLE 9

### In Vitro Release

In vitro release testing is a commonly used technique to examine the performance of topical drug formulations. It is a basic requirement as dictated by regulatory bodies such as the FDA. We utilised a recently developed improved in vitro model that eliminated some pitfalls in previously used methods.

Diffusion experiments were performed using all glass Franz-type cells (nominal receptor phase volume, 3 ml). Nylon sheet membranes were soaked in the receptor medium (appropriate co-solvent mix) for 24 hours prior to commencement. The membranes were then taken out of the receptor fluid, the surface dried and carefully placed onto the pre-greased flange of the receptor compartment. The donor chamber was then placed onto the corresponding receptor compartment and pinch clamped in position. To each receptor compartment a micro stirrer was added. The effectiveness of this technique for PBS and the more viscous PEG400 was previously validated by applying small aliquots of dye to receptor compartments filled with solution.

An infinite dose was added by adding the formulations into each of the appropriate donor caps until the amount of gel added reached the top of the donor chamber. Pre-greased glass cover slips were then placed onto each of the donor chambers to form an occlusive airtight seal. The receptor compartments were then filled with the appropriate degassed co-solvent mix (either 40:40:23 Water:Propylene Glycol:Urea, 50:40:10 PG:ETOH:Water or 10% EtOH/Buffered Solution—salt concentration identical to that present in the ampules) and the cells placed on a multiple stirrer plate in a thermostatically controlled water bath, where the temperature at the surface of the membrane was maintained at 32° C. 200 μl samples were collected at 1, 2, 4, 6, 12 and 24 hrs and replaced with the appropriate temperature-equilibrated co-solvent mix. A total of 6 replicates were carried out for each treatment.

### EXAMPLE 10

### HPLC Analysis

A Dual HPLC-UV analytical method for separation and detection of the 2 activities was developed. HPLC analysis was performed using a Hewlett Packard 1100 HPLC automated system fitted with a Phenomenex Kingsorb 5 μm C18 Column (250×4.6 mm). The mobile phase consisted of 40:30:30 Water:MeOH:MECN. The UV detector was set to 200 nm and a 20 μl injection volume was used. The flow rate was 1 ml min⁻¹, the run time was 10 minutes and the retention time of furosemide was typically 3.2 minutes and Digoxin 5.4 minutes. Standard calibration curves were constructed from standard solutions (range 0.1, 1, 10, 20, 40, 80 and 100 μg ml¹) that contained the relative same proportions of the solvents or co-solvent mixes. A Chromatograph to illustrate the dual HPLC assay for the separation and detection of F (first peak observed at 2.6 minutes) and D (second peak observed at 5.2 minutes) was obtained. This Chromatograph was obtained following an injection of a combined standard solution that contained D and F (both at a concentration of 50 μg ml⁻¹).

Results are indicated in FIGS. 8 to 14.

Summary of the in vitro Release data produced from each gel (taken from FIGS. 8 through 11).

| Gel 14:1 | | | | | | | Gel 1:1 | |
|---|---|---|---|---|---|---|---|---|
| | FUROSEMIDE | | | DIGOXIN | | | FUROSEMIDE | DIGOXIN |
| | pH 5 | pH7 | pH 9 | pH 5 | pH 7 | pH 9 | pH 7 | pH 7 |
| Q₂₄ | 543.60 | 365.65 | 230.69 | 42.47 | 17.62 | 3.34 | 64.63 | 14.07 |
| (μg cm⁻²) | | | | | | | | |
| Flux | 22.65 | 15.24 | 9.61 | 1.77 | 0.73 | 0.14 | 2.69 | 0.59 |
| (μg cm⁻² | | | | | | | | |
| h⁻¹) | | | | | | | | |
| Release | 124.51 | 77.482 | 43.389 | 10.18 | 4.30 | 0.86 | 7.18 | 3.48 |
| Rate | (0.9993) | (0.9974) | (0.9993) | (0.9836) | (0.9961) | (0.9742) | (0.9844) | (0.9981) |
| (μg cm⁻² | | | | | | | | |
| h^{0.5}) | | | | | | | | |
| (R₂) | | | | | | | | |
| % | 8.7 | 5.9 | 3.7 | 4.0 | 1.6 | 0.1 | 17.9 | 1.6 |
| applied | | | | | | | | |
| dose | | | | | | | | |

Summary of in vitro Release data produced from each gel (taken from FIGS. 12 through 14).

| | Ultrez | | HPC | | Ampule | |
|---|---|---|---|---|---|---|
| | Formulation | | Formulation | | Formulation | |
| | Furosemide | Digoxin | Furosemide | Digoxin | Furosemide | Digoxin |
| Q₂₄ | 6128 | 1138 | 9085 | 934 | 2724 | 34 |
| (μg cm⁻²) | | | | | | |
| Release | 1027 | 274 | 2673 | 243 | 667 | 8.1 |
| date | | | | | | |
| (μg cm⁻² h^{0.5}) | | | | | | |
| (R₂) | | | | | | |
| Flux | 248 | 45 | 388 | 39 | 111 | 1.4 |
| (μg cm⁻² h⁻¹) | | | | | | |

Data Processing

| | |
|---|---|
| Q24: | This value is the total concentration of active per unit area (μg |
| | cm⁻²) that has been released into the receptor phase in 24 |
| | hours. |
| Flux: | This is concentration of active that has been release per hour |
| | per unit area (μg cm h−1). Calculated by dividing Q24 by 24. |
| Release Rate: | This is the gradient of the line taken from the Square Root of |
| | Time (hours) plot (μg cm−2 h0.5). The R2 value is also reported |
| | indicating line rarity. |
| % Applied Dose | The amount of active present in the receptor phase calculated |
| | as a percentage of the dose applied. Assuming the donor phase |
| | contains fixed 2 g of gel |

It has become a matter of routine that in vitro studies that investigate percutaneous permeation of topical creams, gels, ointments through human skin typically uses a phosphate buffered saline (PBS) receptor phase. PBS receptor phases are designed to provide similar physiological solute conditions to those within and underlying the dermis. Many other different types of receptor phases have also been employed during in vitro studies, with one individual study utilising four different receptor phases being HBS, HEPES buffer (1.5 mM CaCl₂), HEPES buffer (10% Bovine Calf Serum) and trizma base 45 mM (40 mM sodium cholate).

However, the main purpose of in vitro release studies is to gain quantitative data on the release of the active from the topical formulation.

Both gels exemplified used the same improved in vitro release system, in which the co-solvents contained within the topical formulation and the contents of the receptor phase were both the 40:40:20 Water:Propylene Glycol:Urea gel mix. By employing the 40:40:20 gel mix as the receptor phase instead of the traditional phosphate buffered saline (PBS) co-elution of excipients in the formulation are minimal allowing only net translocation of Furosemide and Digoxin from the donor. Also soaking the membranes in this mix prior to cell assembly ensured that the only net migration would be the actives.

The maximum concentrations of Furosemide and Digoxin observed in the receptor phase were 543.60 μg ml⁻¹, 42.47 μg ml⁻¹ respectively significantly lower than both the saturated solubilities (determined from earlier solubility studies). Therefore sink conditions were maintained throughout the experiments.

When plotted as the square of root time (hours) the release profiles obtained for Furosemide and Digoxin show excellent linear ties. R₂ values calculated were; 0.9993, 0.9974, 0.9993 for Furosemide at pH 5, 7 and 9 from the 1:14 gel, 0.9836, 09961, 0.9742 for Digoxin at pH 5, 7, and 9 from the 1:14 gel, 0.9844 and 0.9981 for Furosemide and Digoxin respectively from the 1:1 gel. This suggests that the nylon release membrane chosen exerted minimal/zero rate limiting effects for both formulation.

Differences in Release Characteristics with Changing pH

For both Furosemide and Digoxin, as pH of the gel increased, there was a rank order reduction in the release rates. Furosemide at pH 5 had a release rate of 124.51 μg cm⁻²h^{0.5}, yet at pH 9 this had been reduced to 43.389 μg cm⁻²h^{0.5} almost a three-fold reduction. The reduction in the rate of release was more substantial for Digoxin at pH 5 10.18 μg cm⁻²h^{0.5} was release but was reduced to 086 μg cm⁻²h^{0.5} at pH 9, a twelve-fold reduction.

Given that Digoxin is a neutral permeant the observations of reduced release with increasing pH was attributed to physiochemical properties of the gel (given that thickening characteristics of the gel can be affected via changes in pH). Furosemide is chemically amphiphillic which may explain the less significant changes in the rates of release with changes in pH.

These results would support formulating the gel at pH 5 to obtain optimum simultaneous release characteristics for both permeants.

### Differences in Release Characteristics Between Gels of Different Permeant Stoichiometry.

The in vitro Release Tests were extended to also investigate the differences between two gels that differed by the relative stoichiometry of Furosemide to Digoxin. The first gel was formulated as described in Rationale 1′ having a 1:1 molar ratio of Digoxin:Furosemide. The second gel was formulated according to ‘Rationale 2’ result in a 1:14 molar ratio of Digoxin:Furosemide. Both gels were formulated at a pH of 7. Significant differences in release of both actives from the gels was apparent.

The 1:14 Gel resulted in release rates of 77.482 μg cm⁻²h^{0.5} for Furosemide and 430 μg cm⁻²h^{0.5}. However, release rates produced the 1:1 Gel were significantly lower for Furosemide at 7.18 μg cm⁻²h^{0.5} and also lower at 3.48 μg cm⁻²h^{0.5} for Digoxin. This is a respective 10.8 fold and 1.2-fold reduction. This observation was attributed to the applied doses of the two formulations.

At twenty four hours the amount of Furosemide and Digoxin release from both revised gel formulations (ULTREZ and HPC) was significantly higher than the corresponding amounts released from the Ampule gel formulation. The revised formulations, at twenty four hours showed similar amounts being released in terms of Digoxin, however the HPC thickened gel formulation released almost 1.5 times more Furosemide than the Ultrez formulation.

When observing the Digoxin release rates for the three gels, the Ultrez formulation showed the highest rate of release with 274 μg cm⁻²h^{0.5}, followed by a similar rate of release from the HPC formulation with 245 μg cm⁻²h^{0.5}, however significantly lower rates of release were gained from the Ampule gel formulation with 8.1 μg cm⁻²h^{0.5}.

In terms of rates of release of Furosemide from the three gels, the highest rate of release was observed from the HPC formulation with 2673 μg cm⁻²h^{0.5} followed by a significantly lower rate of release produced from the ULTREZ formulation with 1527 μg cm⁻²h^{0.5}, again significantly lower rates of release were gained from the Ampule formulation.

In terms of the overall release characteristics exhibited by each of the gels, given that the rates of release for Digoxin from the HPC and Ultrez thickened formulation were similar however Furosemide rates of release were significantly higher in for the HPC gel, overall the HPC thickened gel performed better than the Ultrez gel. The performance of the ampule gel formulation was significantly poor when compared to both revised gel formulations.

It can be concluded from these results that a most preferred formulation for the simultaneous co-release of Furosemide and Digoxin from these formulations would be the 1:14 Digoxin:Furosemide at a pH of 5 in the 50:40:10 co-solvent mix thickened with 8% HPC.

Due to the differences of the co-solvent mixes in terms of their relative keratolytic activity, it is important to conduct in vitro permeation experiments to determine which formulation delivers the most amounts of Digoxin and Furosemide through human callous skin.

### In Vitro ‘Callous Skin’ Permeation

### EXAMPLE 11

Obtaining in vitro permeation data using diffusion cells is a commonly used technique to examine the performance of topical drug formulations.

Permeation experiments were carried out using all glass Franz-type cells incorporating callous plantar skin between a donor and receptor phase. Callous cuttings of approximately 0.6 cm diameter were required to fit the customised ‘franz’ cells used. Consequently, an in vitro permeation study was conducted that used 5 cells per gel treatment, with no more than a total of 15 cells being run simultaneously. To each receptor compartment a micro stirrer was added. An infinite dose of gel (˜1 g) was added to each donor compartment via syringe and PEG400, being used as a receptor phase in each, thereby enabling adequate ‘sink’ conditions, limiting hydrodynamic boundary effects and tissue swelling effects. The applied gel was occluded with glass cover slips and the cells stirred in a thermostatically controlled water bath, maintaining the skin temperature at 32° C. 200 μl samples were collected at 1, 3, 6, 12, 24, 36, 48, 72, 96, 120 and 144 hrs (6 days total) and replaced with temperature-equilibrated receptor phase. A total of five replicates were carried our for each gel. All samples were immediately analysed by HPLC.

Initial in vitro permeation trials were conducted on the following prototype gel:

### 1) Prototype Gel

1:14 (Digoxin:Furosemide) in 40:40:20 (PG:Water:Urea) pH 5 (1.5% Carbomer—98 1 NF)

Subsequent in vitro permeation trials were conducted on the following revised gels:

### 2) Maximum Digoxin Gel

1:14 (Digoxin:Furosemide) in 50:40:10 (PG:EtOH:Water) pH 5 (8% HPC)

### 3) Maximum Furosemide (Optimum Digoxin Gel)

1:14 (Digoxin:Fursoemide) in 50:20:20:10 (PG:EtOH:PEG400:Water) pH 5 (8% HPC)

### 4) Digoxin and Furosemide Ampule Gel

2 g of solution taken from the Dioxin ampule was combined with 2 g of solution taken from the Furosemide ampule and 200 μl of ethanol was added. Again, 8% w/w of HPC was added to thicken the gel.

### Data Processing

The steady state flux of both Digoxin and Furosemide for each gel was calculated from the gradient of the linear section of the cumulative permeation profiles. The breakthrough time was taken from the point at which permeant was first detected in the receptor phase (dependent on the MDL for each permeant). Lag times were calculated by extrapolating the linear section of the cumulative amount permeated (steady state flux) to the X-axis (time).

### Results (Prototype Gel)

FIGS. 16 to 20 illustrate the permeation profiles for Furosemide and Digoxin through callous plantar skin from an infinite dose of gel.

TABLE 1
Summary of the Data taken from the 40:40:20 Prototype gel.

| | | Lag | | Permeation |
|---|---|---|---|---|
| | Breakthrough | time, | Steady state flux | co-efficient |
| Permeant | time, (h) | (h) | (μc cm⁻²hr⁻¹) | (cmhr⁻¹) |
| Furosemide | ~6 | 100 | 13.83 ± 1.8 | 2.2 × 10⁻³ |
| Digoxin | ~36 | 100 | 1.14 ± 0.28 | 1.1 × 10⁻³ |

### Conclusions (Prototype Gel)

It was established in an earlier study that at a pH of 5.5 the formulation displays the greatest release rates for Furosemide and Digoxin. This study assessed the capacity of the formulation to deliver both drugs via permeation through callous skin.

The breakthrough time for Digoxin was approx 36 hrs and for Furosemide approx 6 hrs. It is hypothesised that these differences reflect several things:
1) Relatively higher concentration (hence driving force) of Furosemide in the formulation
2) Lower limit of detection for Digoxin (88 ng) relative to frusemide (3 ng) due to the presence of a weaker chromophore in former
3) Significant Differences in permeant molecular weight (Furosemide 330.7 Digoxin 780.9)
4) Barrier properties of the callous membrane

In terms of the physical properties of the skin it is apparent that this study utilised relatively thick human callous plantar skin with an average thickness of 0.92 mm. We believe this to be the best possible alternative (to actual wart lesion tissue surrounding HPV), to study the permeation of the two permants from a gel formulation. As seen in the previous ‘Swelling Study’, tissue used in this study again was shown to increase in mass, probably due to hydration and resultant swelling of the keratin within the callous plantar skin. The average % increase in weight was shown to be 43%.

Overall, Furosemide exhibited a typical permeation profile through the callous plantar skin (FIG. 16). First order kinetics were observed between 120 and 312 hours from which a steady state of flux of 13.83±1.18 μg cm⁻² hr⁻¹ was calculated. For Digoxin a typical permeation profile was also obtained. Steady state was attained at 144 hours to which a flux of 1.14±0.28 μg cm⁻² hr¹ was calculated (FIG. 17). The relative steady state flux for Furosemide and Digoxin may not be entirely due to the physical characteristics of the permeants but also the relative keratolytic effects of the formulation.

FIG. 19 illustrates the molar permeation of both drugs. It can be seen that the ratio of Furosemide and Digoxin (approx. 10:1) does not differ to any great extent from the molar ratio of the drugs found in the formulation (14:1). The higher concentration of Furosemide in the gel is also mirrored in the relative percentages of drug permeated (FIG. 18). The greater propensity for Furosemide to permeate is expected, given Furosemide has a significantly lower molecular weight relative to Digoxin. This is reflected in the calculated apparent steady state flux values (Table 1). However, this effect may not be solely due to the physical properties of the permeants but may also relate to the relative (and perhaps temporary) pore sizes created by the keratolytic effects of the formulation. The specific diameter/3-dimensional shape of the created pores may allow easier permeation of Furosemide rather than Digoxin relative to the permeant molecular weight.

It can be concluded from these results that a gel formulation containing water, propylene glycol and urea (40:40:20) thickened with 1.5% Carbopol 981 NF at pH 5 will successfully deliver Furosemide and Digoxin through callous skin. These results suggest that topical application of such a gel would be beneficial in controlling the Papiloma virus.

Results from this study provide compelling evidence that Furosemide and Digoxin contained within this specific gel formulation can be delivered through human callous plantar skin and useful for the treatment of skin conditions associated with HPV.

### Results—Revised Gels

FIGS. 21 and 22 illustrate the cumulative permeation profiles for both Furosemide and Digoxin respectively, through callous plantar skin from about 1 g of each of the three different gel formulations.

FIGS. 23 and 24 provide a direct comparison between the cumulative permeation of both Digoxin and Furosemide from the prototype gel and revised maximum Digoxin gel.

TABLE 2a
Summary of the Permeation data for the ‘Max’ Digoxin Gel Formulation

| | | Lag | |
|---|---|---|---|
| | Breakthrough | time, | Steady state flux |
| Permeant | time, (h) | (h) | (μc cm⁻²hr⁻¹) |
| Furosemide | ~1 | 55 | 6.759 |
| Digoxin | ~1 | 18 | 1.015 |

TABLE 2b
Summary of the Permeation data for the ‘Max’ Furosemide Gel
Formulation

| | | Lag | |
|---|---|---|---|
| | Breakthrough | time, | Steady state flux |
| Permeant | time, (h) | (h) | (μc cm⁻²hr⁻¹) |
| Furosemide | ~1 | 60 | 5.900 |
| Digoxin | ~12 | 68 | 0.455 |

TABLE 2c
Summary of the Permeation data for the Dig/Fur Ampule Gel
Formulation

| | | Lag | |
|---|---|---|---|
| | Breakthrough | time, | Steady state flux |
| Permeant | time, (h) | (h) | (μc cm⁻²hr⁻¹) |
| Furosemide | ~1 | 61 | 4.370 |
| Digoxin | ~24 | 82 | 0.069 |

### Conclusions (Revised Gels)

This study assessed the ability of three (revised) gel formulations to simultaneously deliver both Furosemide and Digoxin through human callous skin. To highlight potential differences in the ability of the various gels to deliver the actives through the callous skin, a comparison of the permeation data produced by the revised gel formulations, relative to the original prototype gel formulation was conducted.

From the three formulations studied, greatest delivery of both Furosemide and Digoxin through human callous skin was observed from the ‘Max’ Digoxin gel formulation, followed by the Max Furosemide gel, with lowest Furosemide and Digoxin delivery observed from the Digoxin/Furosemide Ampule gel formulation. See FIGS. 21 and 22.

When comparing the amounts of Digoxin and Furosemide delivered from the ‘Max’ Digoxin gel versus the prototype gel, significant differences can be observed. For the ‘M’ Digoxin gel, at 144 hours 620 μg cm⁻² of Furosemide and 101 μg cm⁻² of Digoxin permeated. However, for the prototype gel, at 144 hours only 574 μg cm⁻² of Furosemide and 30 μg cm⁻² of Digoxin permeated a 1.1 and 3.36 fold reduction respectively.

Additionally, for the original gel prototype, the breakthrough time for Digoxin was approx 36 hours and Furosemide approx 6 hours (see Table 1). However, the breakthrough times gained from the ‘Max’ Digoxin gel formulation were significantly quicker, at 1 hour for both Digoxin and Furosemide.

It is hypothesised that these differences reflect several things:
1) Relatively higher concentration (hence driving force), primarily of Digoxin and also Furosemide present in the formulation
2) Lower limit of detection for Digoxin (880 ng) relative to Furosemide (3 ng), due to the presence of a weaker chromophore in former
3) Significant Differences in permeant molecular weight (Furosemide 330.7 Digoxin 7809)
4) Baffler properties of the callous membrane

In terms of the physical properties of the skin, it is apparent that this study utilised relatively thick human callous plantar skin. We believe this to be the best possible alternative (to actual wart lesion tissue surrounding HPV), to study the permeation of the two permeants from a gel formulation.

Furosemide and Digoxin exhibited ‘typical’ permeation profiles through the callous plantar skin (FIG. 2). For the ‘Max’ Digoxin gel formulation, first order kinetics were observed for Furosemide between 72 and 144 hours, and for Digoxin between 48 and 120 hours from which a steady state of flux of 6.759 and 1.015 μg cm⁻² hr⁻¹ accordingly was calculated.

It can be concluded from these results that a gel formulation containing 50:40:10 Propylene Glycol:Ethanol:Water thickened with 8% HPC enables simultaneous delivery of Furosemide and Digoxin through human callous skin. The permeation data produced from the revised ‘Max’ Digoxin gel formulation highlight significant improvements in the relative permeation characteristics for both permeants, when compared to the original prototype formulation. These results suggest that topical application of such a gel would be beneficial in controlling the Papiloma virus.

Results provide compelling evidence that Furosemide and Digoxin contained within this specific gel formulation can be delivered through human callous plantar skin and useful for the treatment of skin conditions associated with HPV.

In addition to the above embodiments the following additional examples show how other gel formulations could be prepared and used.

As stated above, two particularly effective drugs in this context have been shown to be Digoxin and Furosemide and examples of their 50% plaque Inhibitory Concentrations (IC50) are given below (Table A). The IC50 is an often quoted index of antiviral drug potency useful and convenient when comparing different drugs. Used separately, both Digoxin and Furosemide clearly inhibit the replication of a broad range of viruses.

TABLE A

| | | Digoxin IC50 | Furosemide IC50 |
|---|---|---|---|
| Virus | Host Cell | (ng/ml) | (μg/ml) |
| Adenovirus | A549 | 77 ± 22< | 440 ± 114 |
| Cytomegalovirus | MRC5 | 20 ± 7.5 | 460 ± 114 |
| Varicella-Zoster virus | MRC5 | 20 ± 7.5 | 460 ± 114 |
| Herpes simplex virus | MRC5 | 20 ± 7.5 | 460 ± 114 |
| Herpes simplex virus | BHK21 | 30 | 800 |
| Herpes simplex virus | Vero | 60 | 1000 |

An alternative index of antiviral activity, however, demonstrates the true potency of these drugs. Since our so-called ionic contraviral therapy (ICVT) permits the synthesis of non infectious virus proteins and those proteins cause, in part, the changes in cell pathology (cytopathic effect) that form the basis of IC50 determinations, the potency of these drugs is underestimated by IC50 determinations. An alternative index measures instead the total number of infectious virus particles produced by infected cells.

Using Digoxin, for example, inhibition of Herpes Simplex Virus plaque production of between 40% and 60% i.e. the IC50 effect (upper line on graph; FIG. 25) corresponds to between 90% and 99% inhibition of infectious virus particle production (lower line on graph; FIG. 25).

Using Digoxin and Furosemide individually, each at their IC50, against another virus, namely feline herpesvirus, virus replication is almost completely inhibited (Table B). While the production of infectious virus is reduced by 98.5% (Digoxin) and 99.5% (Furosemide) there remains a low level of virus replication; i.e., 1.5% (Furosemide) and 0.5% (Digoxin).

TABLE B

| | Virus particles | Virus particles | Virus |
|---|---|---|---|
| | per cell | per cell | particles per cell |
| Virus | No Drug | Digoxin IC50 | Furosemide IC50 |
| Feline herpes virus | 50 | 0.75 | 0.25 |

It is possible, however, to effectively eliminate this residual, low level of virus replication by using the drugs in combination. The combined antiviral effect being greater than when the drugs are applied separately; the drugs are synergistic (Table C).

TABLE C

| | | Virus particles per cell |
|---|---|---|
| | Virus particles per | Digoxin |
| Virus | cell No Drug | IC50 and Furosemide IC50 |
| Feline herpes virus | 50 | 0.00001 |

Thus, virus replication is reduced by 99.99999%.

The replication of other viruses is also most effectively inhibited by using the drugs in combination, for example, Varicella Zoster Virus (VZV). It is impossible, however, to quantify the precise number of infectious VZV particles involved since VZV is a highly cell-associated virus. Instead the effects of individual and combined IC50s on virus plaque formation are compared (Table D).

Furosemide and Digoxin, each at their respective IC50s inhibited VZV plaque formation, as expected by about 50%; Furosemide 33/61 plaques and Digoxin 21/61 plaques. However, when both drugs at their IC50s were applied in combination. VZV plaque formation was completely inhibited at the low multiplcity of infection (Low MOI). Indeed, VZV plaque formation was completely inhibited when there was one hundred-fold more infection virus in the test system; the High MOI. Using this index of potency, the drugs were, more than one hundred-fold more potent when applied in combination.

TABLE D

| | High MOI¹ | Intermediate MOI² | Low MOI³ |
|---|---|---|---|
| Control | TNTC | TNTC | 61 |
| Furosemide IC50 | TNTC | TNTC | 33⁴ |
| Digoxin IC50 | TNTC | TNTC | 21⁵ |
| Furosemide IC50 | 0⁸ | 0⁷ | 0⁶ |
| And Digoxin IC50 | | | |

¹100 × Low Multiplicity Of Infection
²10 × Low Multiplicity Of Infection
³Low Multiplicity Of Infection
⁴50% plaque inhibition
⁵50% plaque inhibition
⁶100% plaque inhibition
⁷100% plaque inhibition
⁸100% plaque inhibition

Comparison of the combined effects of fractional IC50s provides another index by which to compare the relative potencies the two drugs alone and in combination. In the example below, using Adenovirus, only one quarter of the IC50 of each drug is sufficient, when used in combination, to elicit the same antiviral effect as the IC50 of either drug alone (FIG. 26).

The same phenomenon maintains with Cytomegalovirus (CMV), another strongly cell-associated virus; when the two drugs are used in combination, only one third of the IC50 of each drug is sufficient to elicit the same antiviral effect as the IC50 of either drug alone (FIG. 27).

In summary, Digoxin and Furosemide are synergistic when applied to ICVT. Due to the unique mechanism of antiviral activity (ICVT), the standard IC50 index undervalues true drug potency although the increased, combined effect remains clear using this index.

Most strikingly, the production of infectious virus is decreased by 99.99999% when the drugs are used in combination.

### The Comparative Solubilities and ICVT-Potencies of Digoxin, Digitoxin and Lanoxin (IV)

1) A series of further experiments were undertaken to provide further evidence pertaining to the suitability of these classes of compounds in ICVT treatment, comparative ‘ICVT-ivities’ (ionic contra-viral therapy-activities). Solutions of Digoxin and Digitoxin were prepared from powder to a concentration of 250 μg per ml in 70% ethanol and their ICVT-ivities compared with the ‘standard’ Digoxin preparation; i.e. IV Lanoxin, which is supplied at 250 μg per ml in 10% ethanol.

The ID₅₀ values of Digoxin prepared from powder and Lanoxin (circles) (FIG. 27A) were very similar, i.e. 60 ng per ml. Digitoxin (squares) appeared to be marginally better with an ID50 of 30 ng per ml.

### 2) Comparative Solubilities

Saturated solutions of Digoxin and Digitoxin (were prepared in 90% ethanol and their ‘ICVT-ivities’ compared with the ‘standard’ Digoxin preparation; i.e. Lanoxin.

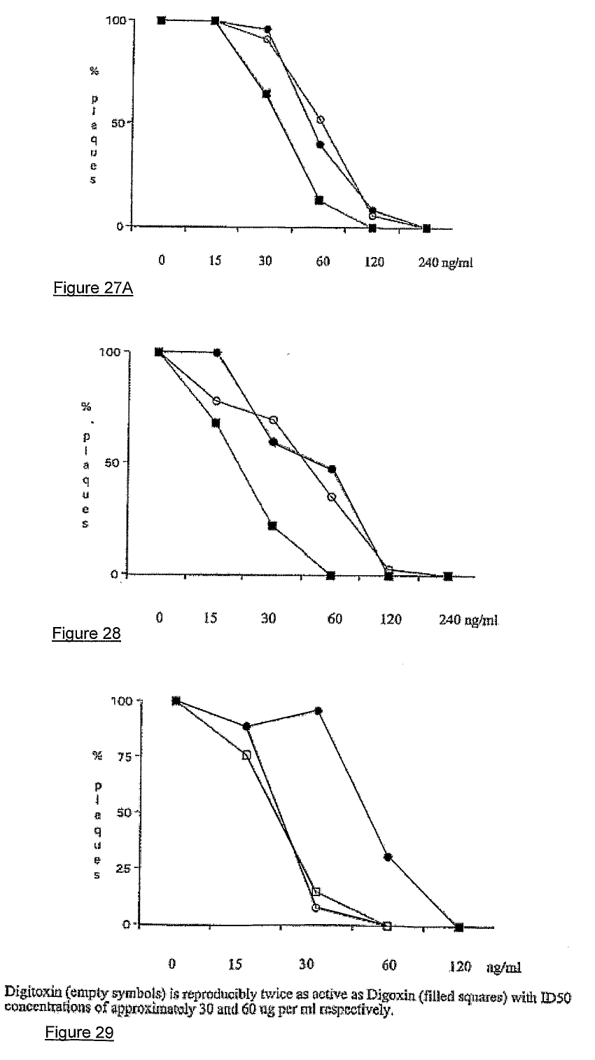

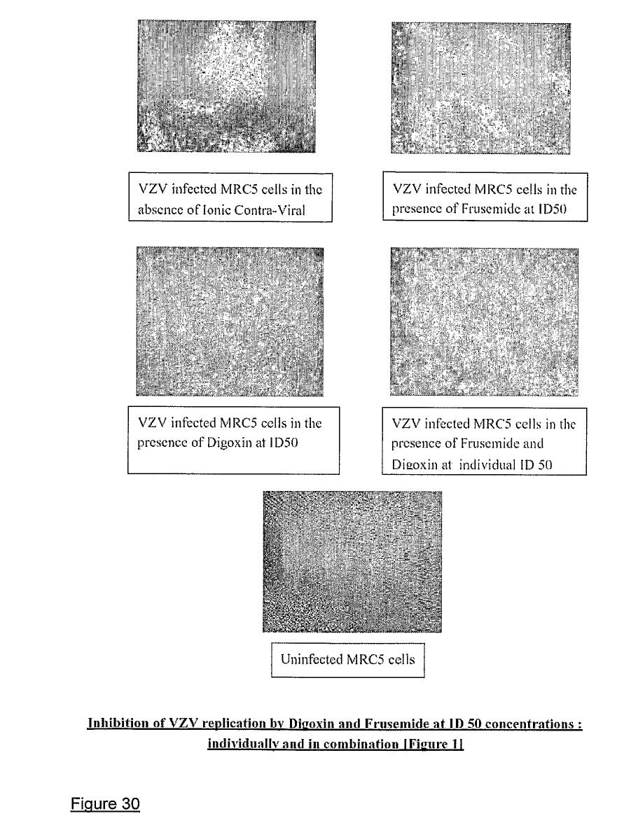

Digoxin solution prepared from powder was as effective as Lanoxin (circles) (FIG. 28).

Digitoxin (squares) was again more effective than Digoxin.

Digitoxin is more soluble than Digoxin; preparation of a saturated solution (17.5 mg per ml) in 90% ethanol will enable use at a maximum concentration of 486 ug per ml in a ‘safe-ocular—concentration (2.5%) of ethanol.

Digoxin was previously used at a concentration of 62.5 ug per ml.

486 ug per ml is approximately eight times more concentrated and if Digitoxin is indeed twice as potent then it might be possible to use what would effectively be 16× the previous ‘dose’. Toxicity at this higher concentration will, of course, need to be examined.

### 3) Comparative Effectiveness

Fresh solutions of Digoxin and Digitoxin were prepared from powder to a concentration of 250 ug per ml in 70% ethanol and their ICVT-ivities again compared with the ‘standard’ Digoxin preparation; i.e. IV Lanoxin in order to further examine their relative potencies. Results are depicted in FIG. 29.

In addition to the above examples, the following further embodiments demonstrate the effects of Frusemide and Digoxin, individually and in combination, on Varicella Zoster virus replication in vitro and on MRC5 cell replication and metabolism.

### 1.1. MRC5

MRC5 cells (Jacobs et al 1970), a line derived from human embryonic lung tissue, were obtained from BioWhittaker. Cells were propagated in Eagles medium (Life Technologies Ltd) supplemented with 10% (v/v) foetal calf serum (Life Technologies Ltd). MRC5 cells were used for Varicella Zoster Virus (VZV) stock production and in experiments investigating the effects of Ionic Contra-Virals on VZV replication.

### 1.2. Cell Morphology

The maximum drug concentration permitting normal cell was determined by incubation of sub-confluent cultures in drug-containing media for 72 hours. Cells were examined directly using phase contrast microscopy.

### 1.3. Cell Replication

The maximum drug concentration permitting cell replication was determined similarly; after 72 hours cells were harvested and counted. A tenfold increase in cell number was taken to be representative of normal cell replication (minimally three population doublings in 72 hours).

### 1.4. MTT (Dimethylthiazol Diphenyltetrtazolium Bromide) Assay

MTT assays were performed as described in Antiviral Methods and Protocols (Kinchington, 2000).

### 1.5. Varicella Zoster Virus (VZV)

The Ellen strain of VZV was obtained from the American Type Culture Collection.

### 1.6. VZV Monolayer Plaque Inhibition Assay

VZV infected cells were assayed on preformed monolayers of MRC5 cells in 5 cm petri dishes by innoculation with 5 ml of infected cell suspension and incubation for 72 hours, or until viral cpe was optimal. Cells were fixed with formol saline and stained with carbol fuchsin.

### 2. Results

### 2.1. The Effect of Frusemide on VZV Replication In Vitro.

Frusemide at a concentration of 1.0 mg/ml was very well tolerated by MRC5 cells; there was no adverse effect on cell morphology and cells replicated. Frusemide inhibited VZV plaque formation by 50% at this concentration.

Frusemide ID 50; 1.0 mg/ml. [Table E]
VZV replication was completely inhibited by Frusemide at a concentration of 2.0 mg/ml.

### 2.2 The Effect of Digoxin on VZV Replication In Vitro

Digoxin at a concentration of 0.05 ug/ml was very well tolerated by MRC5 cells; there was no adverse effect on cell morphology and cells replicated. Digoxin inhibited VZV plaque formation by 50% at this concentration.

Digoxin ID 50; 0.05 ug/ml. [Table E]

VZV replication was completely inhibited by Digoxin at a concentration of 0.1 ug/ml.

### 2.3. The Effects of Frusemide and Digoxin on VZV Replication In Vitro

VZV replication was completely inhibited by Frusemide and Digoxin in combination at their individual ID 50 concentrations [Table E]. The combined dosage was equally well tolerated by MRC5 cells; there was no adverse effect on cell morphology and cells replicated.

### The Effects of Frusemide and Digoxin, Individually and in Combination, on Varicella Zoster Virus Replication In Vitro [Table E]

NB. There was a ten-fold difference between adjacent multiplicities if infection (MOI)

TABLE E

| | HIGH | INTERMEDIATE | LOW |
|---|---|---|---|
| | MOI | MOI | MOI |
| CONTROL | TNTC* | TNTC | 61 |
| Frusemide 0.5 mg/ml | TNTC | TNTC | 33¹ |
| Frusemide 1.0 mg/ml | TNTC | TNTC | 16 |
| Frusemide 2.0 mg/ml | 0² | 0² | 0² |
| Digoxin 0.025 ug/ml | TNTC | TNTC | 55 |
| Digoxin 0.050 ug/ml | TNTC | TNTC | 21³ |
| Digoxin 0.100 ug/ml | 0⁴ | 0⁴ | 0⁴ |
| Frusemide 0.5 ug/ml | 0⁵ | 0⁵ | 0⁵ |
| Digoxin 0.050 ug/ml | | | |

TNTC* Too numerous to count.
¹Frusemide 50% Plaque Inhibitory Dose [ID 50] 0.5 mg/ml.
²Frusemide completely inhibited VZV at a concentration of 2.0 mg/ml.
³Digoxin 50% Plaque Inhibitory Dose ID 50; 0.05 ug/ml.
⁴Digoxin completely inhibited VZV replication at a concentration of 0.1 ug/ml.
⁵VZV replication was completely inhibited by Frusemide and Digoxin in combination at their individual ID 50 concentrations.

### 2.4. The Effect of Frusemide on MRC5 Cell Replication

Uninfected MRC5 cells replicated to normal yields in the presence of Frusemide at a concentration of 1.0 mg/ml, the same concentration as the VZV ID50.

### 2.5. The Effect of Digoxin on MRC5 Cell Replication

Uninfected MRC5 cells replicated to normal yields in the presence of Digoxin at a concentration of 0.05 μg/ml, the same concentration as the VZV ID50.

### 2.6. The Effects of Frusemide and Digoxin on MRC5 Cell Replication

Uninfected MRC5 cells replicated, though not to normal yields, in the presence of both Frusemide and Digoxin at their VZV ID50 concentrations. At these concentrations, VZV replication was completely inhibited.

### 2.7. The Effects of Frusemide and Digoxin on MRC5 Cell Metabolism

The effects of Frusemide and Digoxin on MRC5 cell metabolism were measured using the MTT assay. There were normal levels of metabolism in uninfected cells incubated with either Frusemide or Digoxin at their VZV ID 50 concentrations. There was normal metabolism in uninfected cells incubated with both Frusemide and Digoxin at their VZV ID 50 concentrations. In combination at these concentrations VZV replication was completely inhibited (2.3).

In addition to the above examples, the following further embodiments demonstrate the efficacies of alternative diuretics and cardiac glycosides.

Examples of Thiazide (Hydrochlorothiazide and Metolazone), Sulphonylurea (Tolbutamide), Sulphonamide (Furosemide, Acetazolamide, Bumetanide, Torasemide and Ethacrynic acid) and K sparing diuretic (Amiloride) were tested for ICVT activity. The cardiac glycosides Digoxin, Digitoxin, Lanoxin and Strophanthin G were also tested.

Using Herpes simplex virus (HSV), 50% plaque inhibitory dose (1D50) were established using the standard plaque inhibition assay. Various solvents were required to facilitate testing and these were sometimes detrimental to tissue culture, depending upon their concentration. Certain compounds elicited potent ICVT activity (Furosemide, Digoxin, Lanoxin and Digitoxin) and these were active at high dilution; experimental conditions in which solvent toxicity was excluded.

Other compounds elicited only ‘borderline’ CVI activity. These compounds (Acetazolamide, Tolbutamide and Hydrochiorthiazide) were further tested using alternative solvents in the same test system (ie the plaque inhibition assay) and others (Bumetanide, Torasemide, Tolbutamide and Hydrochlothiazide) in a more sensitive test for ICVT activity in which the effects on virus yields were determined. The effects of cardiac glycosides Digoxin and Strophanthin on virus yields were also tested in this assay.

| | |
|---|---|
| Thiazide | |
| Hydrochiorothiazide | |
| Solvent: Ethanol 10% 5 mg/ml | |
| HSV Plaque 1D50 Negative @ 2.5 mg/ml | − |
| Solvent: NaOH 1% aqueous 1 0 mg/ml | |
| HSV Plaque 1D50 400 ug/ml Borderline | +/ |
| HSV yield reduced to zero at 600 ug/ml | + |
| Metolazone | |
| Solvent: PEG 10 mg/ml | − |
| Solvent: PG 0 mg/ml | − |
| Sulphonylurea | |
| Tolbutamide | |
| Solvent: NaOH 1% aqueous 10 mg/ml | |
| HSV Plaque ID50 500 μg/ml Borderline | +/ |
| Solvent: PEG 10 mg/ml | |
| HSV Plaque 1D50 500 μg/ml Borderline | +/ |
| HSV yield reduced to zero 300 μg/ml | + |
| Solvent: PG 10 mg/ml | |
| HSV Plaque ID50 500 μg/ml Borderline | +/− |
| HSV yield reduced to zero 300 μg/ml | + |
| Solvent IPA 10 mg/ml | |
| HSV Plaque 1D50 250 μg/ml Borderline | +/ |
| Sulphonamide | |
| Furosemide | + |
| Solvent: aqueous (IV) 10 mg/ml | |
| HSV Plaque 1D50 1 mg/ml | |
| Acetazolamide | |
| Sigma | |
| Solvent: PEG 40 mg/ml | |
| HSV Plaque 1D50 Negative @ 500 μg/ml | − |
| Solvent: PG 7 mg.ml | |
| HSV Plaque 1D50 Negative @ 100 μg/ml | − |
| Bumetanide | |
| Solvent: (IV) Aqueous 500 μg/ml | |
| HSV Plaque 1D50 Negative @ 100 μg/ml | − |
| HSV yield reduced Borderline | +/− |
| Torasemide | |
| Qemaco | |
| Solvent: NaOH 1% aqueous 5 mg/ml | |
| HSV Plaque 1D50 60 μg/ml Borderline | +/ |
| HSV yield unaffected at 90 μg/ml | − |
| Ethacrynic acid | |
| Solvent; (IV) Aqueous 100 μg/ml | |
| HSV Plaque 1D50 25 μg/ml Negative | |
| K sparing diuretic | |
| Amiloride | |
| Solvent: Aqueous 500 μg/ml | |
| HSV Plaque ID5O 250 μg/ml | +/− |
| Cardiac glycoside | |
| Digoxin (IV) 250 μg/ml | |
| HSV Plaque 1D50 60 ng/ml | + |
| HSV yield reduced | + |
| Digitoxin | |
| Solvent: Ethanol | |
| HSV Plaque ID50 30 ng/ml | + |
| HSV yield reduced | + |
| Lanoxin (IV) 250 μg/ml | |
| HSV Plaque ID5O 60 ng/ml | + |
| HSV yield reduced | + |
| Strophanthin G | |
| Solvent: Aqueous | |
| HSV Plaque 1D50 1 mg/ml Cytotoxic | |
| HSV yield reduced Borderline | +/− |

The above data shows that other diuretics and cardiac glycosides demonstrate ICVT efficacy. Clearly, the use of other diuretics and cardiac glycosides is possible in the context of gel formulations for topical application for the treatment of DNA viral infections, such as HPV.

1. An anti-viral topical gel formulation comprising at least one diuretic and/or cardiac glycoside in a gel carrier medium, said formulation capable of transdermal delivery of the said diuretic and/or glycoside.

2. A formulation as claimed in claim 1, capable of transcutaneous delivery of the said diuretic and/or glycoside through the stratum corneum to the basal epidermis.

3. A formulation as claimed in either claim 1, wherein the said diuretic and/or glycoside is capable of percutaneous absorption.

4. A formulation as claimed in claim 1, wherein the delivery capability of the gel medium is intercellular, intracellular and/or shunt route.

5. A formulation as claimed in claim 1, in combination with an occlusive dressing, coating or other layer.

6. A formulation as claimed in claim 1, wherein at least one loop diuretic is present in combination with at least one cardiac glycoside.

7. A formulation as claimed in claim 6, in which the loop diuretic comprises one or more of the following: furosemide, bumetranide, ethacrynic acid and torazemide.

8. A formulation as claimed in claim 7, in which the diuretic is furosemide.

9. A formulation as claimed in claim 1, wherein the cardiac glycoside comprises one or more of the following: digoxin, digitoxin, meDigoxin, lanatoside C, proscillaridin, k strophantin, peruvoside and ouabain.

10. A formulation as claimed in claim 9, wherein the cardiac glycoside is digoxin.

11. A formulation as claimed in claim 1, in which the bioavailability of the diuretic and/or glycoside is such as to provide a steady state flux in the range of 0.1 to 50 μg/cm²/hour; preferably 0.2 to 40 μg/cm²/hour; more preferably 0.5 to 30 μg/cm²/hour; most preferably 1 to 20 μg/cm²/hour.

12. A formulation as claimed in claim 1, wherein the gel medium is a two phase medium structured matrix comprising discrete particles of glycoside and/or diuretic in a semi-solid medium having predominantly a liquid phase, with or without thickener.

13. A formulation as claimed in claim 1, further comprising at least one excipient which is skin-tolerant and/or keratolytic.

14. A formulation as claimed in claim 13, wherein the said excipient comprises urea in an amount in the range of 5 to 30% by weight, preferably 10 to 25%; more preferably 15 to 25% by weight; most preferably about 20% by weight.

15. A formulation as claimed in claim 1, in which the gel carrier medium comprises a cosolvent mix.

16. A formulation as claimed in claim 1, wherein the molar ratio of the glycoside:diuretic is in the range of 0.1 to 10:30 to 0.1, preferably 0.2 to 5:20 to 0.2, more preferably 0.5 to 2.5:20 to 0.5, most preferably 0.5 to 1.5:20 to 10 such as 1:14 to 1.

17. A formulation as claimed in claim 1, in which the gel carrier medium comprises at least one of the following:
i) an alkylene glycol, such as propylene glycol;
ii) water;
iii) a monohydric alkanol, such as an alkanol with up to 4 carbon atoms; and
iv) a polyalkylene glycol, such as polyethylene glycol

18. A formulation as claimed in claim 17, wherein two or more of components (i) to (iv) as defined in claim 17 are present.

19. A formulation as claimed in claim 17, wherein three or four of components (i) to (iv) as defined in claim 17 are present.

20. A formulation as claimed in claim 17, wherein the amount of (i) if present is in the range of 40% to 70% by volume, the amount of (ii) if present is in the range of up to about 40% by volume, and the amount of (iv) if present is in the range of up to about 40% by volume, wherein the % amounts expressed by volume are based on the volume of that component individually as a percentage of the total formulation.

21. A formulation as claimed in claim 17, wherein (iv) polyethylene glycol is present of average molecular weight in the range of 100 to 1000, preferably 150 to 800, more preferably 200 to 600, most preferably 300 to 500 such as about 400.

22. A formulation as claimed in claim 1, in which the carrier medium comprises (i) propylene glycol, (ii) sterile water and (iii) urea.

23. A formulation as claimed in claim 1, wherein the gel carrier medium comprises (i) propylene glycol, (ii) sterile water and (iii) ethanol.

24. A formulation as claimed in claim 23, further comprising (iv) polyethylene glycol of average molecular weight in the region of 400.

25. A formulation as claimed in claim 1, in which the pH is less than 7.

26. A formulation as claimed in claim 25, in which the pH is less than 6.

27. A formulation as claimed in claim 1, wherein the pH is no higher than 3, preferably no higher than 4, most preferably no higher than 5.

28. A formulation as claimed in claim 1, in which the formulation comprises (vi) one or more thickeners.

29. A formulation as claimed in claim 28, in which the thickener comprises one or more of the following: Carbomers such as Carbopol or Ultrez or cellulose derivatives such as hydroxyalkylcellulose.

30. A formulation as claimed in claim 29, wherein the thickener comprises hydroxypropylcellulose, preferably in an amount of 5 to 15% by weight based on the total weight of the formulation.

31. A formulation as claimed in claim 17, which comprises:
(i) an alkylene glycol;
(ii) water;
(iii) a monohydric alcohol, and optionally
(iv) a polyalkylene glycol.

32. A formulation as claimed in claim 31, in which (iv) a polyalkylene glycol is present.

33. A formulation as claimed in claim 31, comprising:
(i) propylene glycol;
(ii) ethanol;
(iii) water, and optionally
(iv) polyethylene glycol of average molecular weight in the range of 200 to 600.

34. A formulation as claimed in claim 33, comprising: 20-60 parts by weight of (i) propylene glycol: 5-60 parts by weight of water: 10-50 parts by weight of ethanol: 0-20 parts by weight of polyethylene glycol of average molecular weight in the range of 200 to 600.

35. A formulation as claimed in claim 31, wherein the molar ratio of cardiac glycoside:loop diuretic is such that molar excess of loop diuretic is present.

36. A formulation as claimed in claim 35, wherein the molar ratio of cardiac glycoside:loop diuretic is in the range of 1:10-20, such as 1:12-18.

37. A formulation as claimed in claim 30, comprising a carbomer thickener in an amount of 0.5 to 5% by weight, based on the total weight of the formulation.

38. A formulation as claimed in claim 1, further including at least one of the following: emulsifier, antioxidant, propellant, colour, buffer, preservative and adhesive.

39. A formulation as claimed in claim 1, for use in the treatment of DNA viral infections.

40. A formulation as claimed in claim 39, for use in the treatment of human papilloma virus infection; such as latent, sub-clinical or clinical HPV infection.

41. A formulation as claimed in claim 1, for use in the topical treatment of warts.

42. A formulation as claimed in claim 1, for use in the reduction or prevention of viral replications by reduction or depletion of viral intracellular potassium ions.

43. A formulation as claimed in claim 1, for use in the preparation of a medicament for use in treating DNA virus.

44. A formulation as claimed in claim 43, in which the DNA virus is human papilloma virus.

45. A formulation as claimed in claim 43, for use in the preparation of a medicament for use in topical application to warts.

46. A formulation as claimed in claim 43, for use in the preparation of a medicament for use in reducing or depleting intracellular potassium ions.

47. A method of treating DNA viral infections, the method comprising applying a topical composition to a subject, wherein the topical composition comprises a formulation as claimed in claim 1.
